(19) United States
(12) Patent Application Publication
(10) Pub. No.: US 2026/0194428 A1
(43) Pub. Date: Jul. 9, 2026

(54) ANALYZING CARBON ISOTOPES OF METHANE PRODUCED BY MICROORGANISMS AFTER EXOGENOUS CARBON ADDITION TO GEOLOGIC FORMATIONS

(71) Applicant: Transworld Technologies Inc., Golden, CO (US)

(72) Inventors: Gary VANZIN, Golden, CO (US); Roland P. DEBRUYN, Golden, CO (US); Joseph Edward ZEMETRA, Golden, CO (US)

(73) Assignee: Transworld Technologies Inc., Golden, CO (US)

(21) Appl. No.: 19/130,876

(22) PCT Filed: Nov. 16, 2023
(86) PCT No.: PCT/US2023/080043

§ 371 Date: May 16, 2025

Related U.S. Application Data
(60) Provisional application No. 63/426,026, filed on Nov. 16, 2022.

(51) Int. Cl. G01N 1/40; G01N 33/00

(52) U.S. Cl. CPC G01N 1/4044; G01N 33/0055

(57) ABSTRACT

Described are techniques for distinguishing between biomethane and fossil methane using isotopic analysis. Examples include evaluating a methane source using radioisotope analysis stable isotope analysis. In some cases, radioisotope analysis is used for purposes of calibrating a stable isotope analysis technique or for verifying precision or reliability of a stable isotope analysis result.

### CROSS-REFERENCE TO RELATED APPLICATIONS

This application claims the benefit of and priority to U.S. Provisional Application No. 63/426,026, filed on Nov. 16, 2022, which is hereby incorporated by reference.

### FIELD

This invention is in the field of renewable natural gas. This invention relates generally to subterranean biological methane generation and identification.

### BACKGROUND

Increasing world energy demand is creating unprecedented challenges for recovering energy resources and mitigating the environmental impact of using those resources. Some have argued that the worldwide production rates for oil and domestic natural gas will peak within a decade or less. Once this peak is reached, primary recovery of oil and domestic natural gas will start to decline, as the most easily recoverable energy stocks start to dry up. Historically, old oil fields and coal mines are abandoned once the easily recoverable materials are extracted.

As worldwide energy prices continue to rise, it may become economically viable to extract additional oil and coal from these formations with conventional drilling and mining techniques. However, a point will be reached where more energy is required to recover the resources than can be gained by the recovery. At that point, traditional recovery mechanisms will become uneconomical, regardless of the price of energy.

Meanwhile, renewable energy sources are being developed and have started to reach price-parity with fossil energy sources. Although renewable energy sources are useful in place of fossil fuel energy, energy distribution infrastructure (e.g., pipelines, etc.) is already in place for transporting/delivering fossil hydrocarbon fuels, and so it is desirable to generate renewable fuels, such as biomethane, which can use the existing infrastructure. Various processes exist for generating renewable fuels, but additional advances are still needed.

### SUMMARY

Provided herein are methods, systems, and techniques for generating renewable fuels, such as biomethane, and for analyzing the produced fuels to determine amounts of the produced fuels that correspond to the generated renewable fuels. Methods, systems, and techniques described herein include those in which renewable methane (biomethane) is generated in a geologic formation by a consortium of symbiotic methanogenic microorganisms anaerobically digesting an injected biogenic feedstock, such as a liquid comprising waste glycerol, erythritol, sorbitol, or maltitol or other waste products (e.g., wastewater, a carbonaceous material source, or the like); the generated biomethane is produced from the geologic formation as produced methane or natural gas and subjected to an analysis to determine an amount (e.g., a fraction) of the methane or natural gas that is biogenic, that is, generated by the consortium of symbiotic methanogenic microorganisms from the biogenic feedstock. In examples, the analysis can involve isotopic analyses, such as stable isotope analysis and/or radioisotope analysis. The radioisotope analysis can be used to establish a baseline amount of the methane or natural gas that is biogenic and can be used in tandem with a stable isotope analysis for verification or calibration of the stable isotope analysis for later use.

Advantageously, and in examples, methods described herein are useful for generating and evaluating methane extracted from a geologic formation, such as an oil field reservoir, such as to determine the amount of methane produced from the reservoir that is biogenic in origin. Many or most oil field reservoirs, even depleted oil field reservoirs, contain existing hydrocarbons, including methane, that are exclusively fossil in origin. It will be appreciated that many oil field reservoirs lie deep below the surface of the earth, such as at a depth where the reservoir does not interact with or is completely or practically isolated or confined from the surface, groundwater, or other aquifer interacting with the surface, and therefore contain little, if any biogenic carbon, and practically contain only fossil carbon which may be derived from the larger hydrocarbons (e.g., oil) present in the reservoir. In examples, such oil field reservoirs are useful as locations for anaerobic digestion of a feedstock by a consortium of microorganisms to generate methane or other gaseous products. Such generated methane will mix with existing methane in the reservoir and, thus, methane produced from the reservoir may be a combination of the original fossil methane and the methane produced by anaerobic digestion of the feedstock. When the feedstock is a biogenic or renewable feedstock, the methane produced by anaerobic digestion then corresponds to biogenic or renewable methane. With a goal of converting renewable carbon containing feedstocks in the subsurface to renewable methane, it may be desirable to use feedstocks with a much greater thermodynamic likelihood of being converted to methane over fossil carbon residing in the reservoir. Nonetheless, in some examples, stimulating native microbial consortia through the introduction of labile feedstocks may also simulate them to metabolize native, fossil hydrocarbons to methane. Thus, it can be useful to differentiate renewable carbon in methane from fossil carbon in methane, and the methods, systems, and techniques disclosed herein provide for such differentiation.

The techniques described herein provide for the generation and characterization of the produced methane by isotopic analysis techniques, such as stable isotope analysis and radioisotope analysis, to determine the amount or fraction of the produced methane that is generated by the anaerobic digestion and corresponds to renewable methane. Advantageously, the disclosed techniques allow for evaluation using stable isotope analysis, which can be faster and more efficient than radioisotope analysis. The techniques described allow the methane to be characterized based on isotopic signatures, such as a ¹³C signature, a ¹⁴C signature, or a combination of both ¹³C and ¹⁴C, for example. Further, the techniques described allow for characterization of the amount of a gaseous product produced by anaerobic digestion of a feedstock that is biogenic in origin without any type of isotopic labeling of the feedstock and/or without enrichment of the feedstock with one or more isotopes. Stated another way, the techniques described can use feedstocks containing natural isotopic abundances, such as where no artificial alteration of isotopic composition in the feedstock is used.

In an aspect, methods for producing and analyzing a fluid are disclosed. An example method of this aspect comprises injecting a feedstock (e.g., a waste stream, a biogenic feedstock, etc.) into a geologic formation, the geologic formation containing a consortium of symbiotic microorganisms, where the consortium of symbiotic microorganisms at least partly anaerobically digests the feedstock to generate a gaseous product, such as a feedstock that contains a known amount or fraction of a radioisotope; producing a fluid from the geologic formation, the fluid comprising the gaseous product, and optionally other gases, such as Ar or N₂; and performing a radioisotope analysis of at least a portion of the fluid, such as the gaseous portion, using the known amount or fraction of the radioisotope, to identify an amount or fraction of the gaseous product in the fluid derived from the feedstock. In this way, the radioisotope analysis can be used to establish an amount or a relative amount or fraction of the gaseous product in the fluid that is biogenic in origin. In some examples, the radioisotope comprises ¹⁴C. In some examples, the gaseous product comprises CH₄, CO₂, H₂, or any combination of these. In examples, the disclosed techniques can use isotopic signatures for methane as a fingerprint to determine the origin—biogenic or fossil—of the methane. Optionally, methods of this aspect may comprise establishing a consortium of microorganisms in a geologic formation, such as by providing an inoculum of the microorganisms into the geologic formation.

In some the examples, the feedstock comprises waste glycerol, erythritol, sorbitol, maltitol, wastewater, a waste product, a carbonaceous material source or the like. Waste products that may be used in some embodiments of the present technology may come from any number of sources that produce wastewater, effluent streams, waste products, or organic wastes having a carbonaceous material content, and which may be consumed by microorganisms within the formation environment. As examples, waste products or carbonaceous material sources may include biodiesel wastewater or agricultural wastewater, however the present technology may use wastewater or waste streams, which may all be encompassed as waste products, including carbonaceous material from a wide variety of sources. The present technology may utilize any type of waste products including developed waste streams in which waste materials are mixed or solubilized in water, or effluent waste streams produced from one or more other activities. In some examples, waste streams or feedstocks may contain carbonaceous materials from a single source or a variety or mixture of sources, such as carbonaceous materials that are from one or more biogenic or renewable carbon sources or other sources. Advantageously, the techniques described herein are useful for determining the amount or fraction of gaseous products, generated from processing of the feedstock, that are biogenic or renewable in origin, regardless of the source of the feedstock.

As non-limiting examples, the present technology may utilize one or more waste product streams including wastes from agriculture, horticulture, aquaculture, forestry, hunting, or fishing; wastes from preparation and processing of meat, fish, dairy, or other foods of animal origin, such as effluent streams from slaughterhouses or commercial food production; wastes from fruit, vegetables, vegetation, cereals, edible oils, cocoa, coffee, tea, or tobacco preparation and processing; conserve production, yeast and yeast extract production, molasses preparation and fermentation; wastes from sugar processing; carbon-containing wastes from refineries or other manufacturing plant processes; wastes from dairy products industry, such as whey; wastes from the baking and confectionary industry; wastes from production or fermentation of alcoholic and non-alcoholic materials or beverages, such as including vinegar production; wastes from wood processing and the production of panels and furniture; wastes from pulp, paper, or cardboard production and processing; wastes from the textiles industry, which may include grease, wax, or other materials; wastes from the manufacture, formulation, supply, or use of basic organic chemicals, and which may or may not include glycerol or other alcohol residues (e.g., glycerol, erythritol, sorbitol, or maltitol); wastes from the aerobic treatments of wastes, such as a non-composted fraction of municipal or similar wastes; wastes from the anaerobic treatment of wastes, such as liquor from anaerobic treatment of municipal waste or similar wastes; grease or oil mixtures from oil/water separation containing edible oils or fats; garden and park wastes, such as including cemetery waste; or any other waste, residual, or effluent material developed during other material processing. In some cases, waste product streams, such as the above, may be used alone or as a combination of waste streams.

Waste products according to some embodiments may include semi-solid waste products including wet solids that may remain from filtration or as solids previously entrained, such as from production of food products, for example. As non-limiting examples, vegan or vegetarian-substitute products may be produced from processed feedstocks, including nuts, soy, or plant materials, and residual solids or semi-solid waste materials or wet streams may be incorporated into waste product streams for delivery into the formation environment. Moreover, waste product streams may be developed from renewable biomass products or wastes. As non-limiting examples, renewable biomass products may include material or waste products from planted crops or crop residue, and which may include all annual or perennial agricultural crops from existing agricultural land that may be used as feedstock for renewable fuel, such as grains, oilseeds, or sugarcane, as well as energy crops, such as switchgrass, prairie grass, duckweed, or other planted, ponded, or grown species. Similarly, crop residue may include the biomass left over from the harvesting or processing of planted crops from existing agricultural land or any biomass removed from existing agricultural land that facilitates crop management including biomass removed from such lands in relation to invasive species control or fire management, whether or not the biomass includes any portion of a crop or crop plant. Renewable biomass may include planted trees or tree residue including slash and any woody residue generated during the processing of planted trees from actively managed tree plantations for use in lumber, paper, furniture or other applications. Renewable biomass may include slash and pre-commercial thinnings, biomass obtained from the immediate vicinity of buildings or other areas regularly occupied by people, or of public infrastructure, at risk from wildfire, algae, as well as materials or waste products identified above including animal waste material and animal by-products or separated yard waste or food waste, including recycled cooking or trap grease, dairy and swine manure, landfill, wastewater or wastewater sludge, food waste, green waste, urban landscaping waste, or other organic waste.

In some examples, the waste streams that are used with the techniques described may include waste streams that are undesirable or unsuitable for use in other processing, such as due to the chemical makeup of the waste. In some examples, waste streams that are acidic may not be suitable for some processing applications, but may still be useful for the techniques described herein without augmenting or changing the pH of the stream. In some examples, waste streams that are basic may not be suitable for some processing applications, but may still be useful for the techniques described herein without augmenting or changing the pH of the stream. In some examples, waste streams that contain excess solutes (e.g., salts) may not be suitable for some processing applications, but may still be useful for the techniques described herein without augmenting or changing the amount of solute (e.g., salinity) of the stream. As one example, a waste stream, such as salt whey, may contain excess salt beyond that desirable for use in certain applications, but such a waste stream may still be useful as a feedstock according to the disclosed methods and systems. For example, when the geologic formation contains water that has considerable salt levels, the addition of a salt-containing waste stream may actually end up diluting the water present in the formation, though in other cases the formation water may be less salty compared to a salt-containing feedstock. In either case, the salt-containing waste stream may still be useful for the techniques described herein, even though such a waste stream may not be useful for other, more salt-sensitive applications.

In some examples, a method of this aspect may comprise or further comprise using the amount or fraction of the gaseous product in the fluid derived from the feedstock to determine one or more stable isotope analysis calibration factors. For example, the one or more stable isotope analysis calibration factors may be used in subsequent analyses on the same geologic formation or on another geologic formation, such as to determine the amount or fraction of the gaseous product in a produced fluid that is biogenic in origin using a stable isotope analysis. In some examples, the one or more stable isotope analysis calibration factors include one or more isotopic fractionation factors or are characteristic of or derived from one or more isotopic fractionation factors.

In an example, a method of this aspect may comprise or further comprise injecting a second feedstock into a second geologic formation, the second geologic formation containing a second consortium of symbiotic microorganisms, where the second consortium of symbiotic microorganisms at least partly anaerobically digests the second biogenic feedstock to generate the gaseous product; producing a second fluid from the second geologic formation, the second fluid comprising the gaseous product; and performing a stable isotope analysis of the second fluid, using the one or more stable isotope analysis calibration factors, to determine a second amount or fraction of the gaseous product in the second fluid derived from the second biogenic feedstock.

Optionally, the second geologic formation is the same as the geologic formation or it may be different. Optionally, the second consortium of symbiotic microorganisms may be the same as the consortium of symbiotic microorganisms or it may be different. Optionally, the second biogenic may be the same as the feedstock or it may be different. Optionally, the second fluid may be the same as the fluid or it may be different. Such a process can amount to first using a geologic formation for determination of stable isotope analysis calibration factors and then subsequently using it for processing and evaluation directly using stable isotope analysis. In some cases, radioisotope analysis can be repeated one or more times, such as to verify or update stable isotope analysis calibration factors. It may be desirable to repeat a radioisotope analysis, such as in the event that chemistry within the geologic formation changes. For example, in the event a composition of the feedstock changes, it can be useful to redetermine the stable isotope analysis calibration factors. In another example, in the event a temperature within the geologic formation changes or is identified, it can be useful to redetermine the stable isotope analysis calibration factors. In another example, in the event a composition of the consortium of microorganism changes or is identified, it can be useful to redetermine the stable isotope analysis calibration factors.

In some examples, using the amount or fraction of the gaseous product in the fluid derived from the feedstock to determine the one or more stable isotope analysis calibration factors comprises determining an isotopic distribution for stable isotopes included in the feedstock; determining an isotopic distribution for the stable isotopes included in the gaseous product in the fluid; and determining the one or more stable isotope analysis calibration factors using the isotopic distribution for the stable isotopes included in the biogenic feedstock, the isotopic distribution for the stable isotopes included in the gaseous product in the fluid, and the amount or fraction of the gaseous product in the fluid derived from the feedstock. In examples, the stable isotopes included in the feedstock include ¹²C or ¹³C (as well as ¹H and ²H). In examples, the stable isotopes included in the gaseous product include ¹²C or ¹³C (as well as ¹H and ²H).

In some examples, an isotopic distribution for the stable isotopes included in one or more samples of fluid produced from the geologic formation may be determined prior to injecting any biogenic feedstock, such as to obtain baseline levels. The isotopic distribution for the stable isotopes included in the one or more samples of fluid produced from the geologic formation may optionally be used to determine the one or more stable isotope analysis calibration factors. In other examples, an isotopic distribution and/or baseline levels of gas present in the geologic formation prior to injecting biogenic feedstock is not determined. In some examples, samples of fluid produced from the geologic formation may be obtained while injecting or after (e.g., shortly after, such as within 1 day) injecting a biogenic feedstock. Optionally, such samples can be subjected to stable isotope analysis to determine a baseline level of gas present in the geologic formation at the time of or after injecting the biogenic feedstock.

Other methods of this aspect may bypass using a radioisotope analysis and may instead use one or more stable isotope analysis calibration factors with a stable isotope analysis for producing and analyzing a fluid. In some examples, the stable isotope analysis calibration factors can be determined using a radioisotope analysis, as described above. An example method of this aspect comprises identifying one or more stable isotope analysis calibration factors; injecting a biogenic feedstock into a geologic formation, the geologic formation containing a consortium of symbiotic microorganisms, where the consortium of symbiotic microorganisms at least partly anaerobically digests the biogenic feedstock to generate a gaseous product; producing a fluid from the geologic formation, the fluid comprising the gaseous product; and performing a stable isotope analysis of at least a portion of the fluid (e.g., a gas phase portion), using the one or more stable isotope analysis calibration factors, to determine an amount of the gaseous product in the fluid derived from the biogenic feedstock. In some examples, the one or more stable isotope analysis calibration factors include one or more isotopic fractionation factors or are characteristic of or derived from one or more isotopic fractionation factors. Optionally, radioisotope analysis can be performed periodically or as needed to confirm, update, or otherwise obtain stable isotope analysis calibration factors or derived values from the stable isotope analysis calibration factors (e.g., isotopic fractionation factors).

In some examples, performing the stable isotope analysis of at least a portion of the fluid comprises determining an isotopic distribution for stable isotopes included in the biogenic feedstock; determining an isotopic distribution for the stable isotopes included in the gaseous product in the fluid; and determining the amount or fraction of the gaseous product in the fluid derived from the biogenic feedstock using the isotopic distribution for the stable isotopes included in the biogenic feedstock, the isotopic distribution for the stable isotopes included in the gaseous product in the fluid, and the one or more stable isotope analysis calibration factors.

In some examples, an isotopic distribution for the stable isotopes included in one or more samples of fluid produced from the geologic formation may be determined prior to injecting any biogenic feedstock, such as to obtain baseline levels. The isotopic distribution for the stable isotopes included in the one or more samples of fluid produced from the geologic formation may optionally be used to determine the amount or fraction of the gaseous product in the fluid derived from the biogenic feedstock.

Optionally, identifying the one or more stable isotope analysis calibration factors comprises injecting a second biogenic feedstock into a second geologic formation, the second geologic formation containing a second consortium of symbiotic microorganisms, where the second consortium of symbiotic microorganisms at least partly anaerobically digests the second biogenic feedstock to generate the gaseous product, such as where the second biogenic feedstock contains a known amount of a radioisotope; producing a second fluid from the second geologic formation, the second fluid comprising the gaseous product; and performing a radioisotope analysis of the second fluid, using the known amount of the radioisotope, to determine the one or more stable isotope analysis calibration factors. Optionally, the second geologic formation is the same as the geologic formation or it may be different. Optionally, the second consortium of symbiotic microorganisms may be the same as the consortium of symbiotic microorganisms or it may be different. Optionally, the second biogenic feedstock may be the same as the biogenic feedstock or it may be different. Optionally, the second fluid may be the same as the fluid or it may be different.

In another aspect, systems for producing and analyzing a fluid are disclosed. An example system of this aspect comprises one or more pumps in fluid communication with a geologic formation containing a consortium of symbiotic microorganisms, such as one or more pumps configured to inject a biogenic feedstock into the geologic formation, where the consortium of symbiotic microorganisms at least partly anaerobically digest the biogenic feedstock to generate gaseous product, and produce a fluid from the geologic formation, the fluid comprising the gaseous product. In some examples, systems of this aspect include the geologic formation containing a consortium of symbiotic microorganisms. In some examples, systems of this aspect include a computing device or a processor in data communication with a non-transitory storage medium comprising executable instructions that, when executed by the computing device or processor, cause the computing device or processor to perform operations including identifying an isotopic distribution for stable isotopes and/or radioisotopes included in the biogenic feedstock, identifying an isotopic distribution for the stable isotopes and/or radioisotopes included in the gaseous product in the fluid, and determining an amount or fraction of the gaseous product in the fluid derived from the biogenic feedstock using the isotopic distribution for the stable isotopes and/or radio isotopes included in the biogenic feedstock, the isotopic distribution for the stable isotopes and/or radio isotopes included in the gaseous product in the fluid, and/or one or more stable isotope analysis calibration factors.

In some examples, the computing device or processor may be configured or programmed with instructions to control the one or more pumps, such as to control an injection rate or a production rate. In some examples, the computing device or processor may be configured or programmed with instructions to control the one or more pumps, such as based on one or more of the isotopic distributions for stable isotopes and/or radio isotopes included in the biogenic feedstock, the isotopic distribution for the stable isotopes and/or radio isotopes included in the gaseous product in the fluid, or the amount or fraction of the gaseous product in the fluid derived from the biogenic feedstock. In some examples, the computing device or processor may be configured or programmed with instructions to identify an isotopic distribution by determining the isotopic distribution or by receiving the isotopic distribution, such as by user input or by wired or wireless data communication. Optionally, the one or more stable isotope analysis calibration factors include one or more isotopic fractionation factors or are characteristic of or derived from one or more isotopic fractionation factors. Optionally, the one or more stable isotope analysis calibration factors are determined using a radioisotope analysis technique.

In some examples, the geologic formation may be a coal bed or other coal-bearing formation or an oil field or other oil-, gas-, or petroleum-bearing formation. The techniques described herein may be useful for coal bed formations as such formations may have significant amounts of water present and so injecting the biogenic feedstock into a coal bed formation can allow the biogenic feedstock to penetrate to any region where the water present may exist, though there may be limited understanding of how the water moves or mixes within the formation. Further, the coal present may bind and adsorb a variety of materials, though not only limited to those generated by microbial action.

Use of the techniques described herein may be further advantageous for use in oil field formations, as such formations often contain oil saturated sand, shale, or sandstone, in addition to brine or other aqueous mixtures in the formation. Many oil field formations may be structured as waterflood formations, where an injection well and a production well are separated by some distance, but the flow of fluids and mixing of fluids though the formation between injection and production can be very well characterized and controlled. Such techniques can allow for improved production of methane from a biogenic feedstock in an oil field formation as compared to a coal bed formation.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a schematic illustration of a geologic formation containing a consortium of symbiotic microorganisms.

FIG. 2 provides an overview of an example method of producing and analyzing a fluid from a geologic formation.

FIG. 3 provides laboratory data showing relationship between δ¹³C and methane for different carbon sources.

FIG. 4 provides a plot of example of data for use in determining stable isotope analysis calibration factors for use in determination of amount of a gaseous product that is biogenic in origin based on stable isotope analysis measurements.

### DETAILED DESCRIPTION

Geologic formations, such as those containing fossil fuels, are increasingly becoming depleted, for example by mining or extraction of the fossil fuels. These depleted formations can be ideal places to generate and/or store large amounts of renewable fuels, like biomethane or biohydrogen, and/or for carbon sequestration (e.g., in the form of carbon dioxide, CO₂). Since the amount of oxygen in a geologic formation can be limited, such formations can be useful as anerobic digesters, where large amounts of methane (CH₄), and optionally CO₂ and/or molecular hydrogen (H₂), can be generated and/or stored.

In some examples, a consortium of symbiotic methanogenic microorganisms may be present in a geologic formation, or may be inoculated into a geologic formation. These microorganisms can be fed a carbonaceous feedstock to generate biomethane. When the carbonaceous feedstock is a biogenic feedstock (e.g., of biogenic origin or derived from living organisms), the biomethane can be a null contributor to the global carbon budget. For example, the biogenic feedstock can be derived from photosynthesis, such as where CO₂ is reduced to generate the biogenic feedstock, effectively removing carbon from the atmosphere, at least temporarily. As that biogenic feedstock is manipulated to produce biomethane, CO₂ can be generated from combustion and/or oxidation of the biomethane, and the carbon can be effectively returned to the atmosphere in the form of CO₂.

Storage of gaseous fuels, such as natural gas, in subterranean reservoirs has been performed in the past. Biofuel storage in subterranean reservoirs, however, does not appear to have been significantly adopted. One concern is that the stored biofuels may mix with fossil fuels in the subterranean reservoirs, particularly when the subterranean reservoir is a fossil fuel reservoir (e.g., a depleted natural gas or oil reservoir). Similarly, when subterranean reservoirs are used directly as anaerobic digesters, any biomethane that is produced by the digestion process may mix with fossil methane present. In such cases, it can be difficult to determine whether any gaseous fuel produced from such a subterranean reservoir is of biogenic origin (e.g., a biofuel) or is a fossil fuel. As climate change becomes an increasing concern, proving that a produced gaseous fuel is of biogenic origin is increasingly desirable. Techniques are described herein where isotopic analysis is used to affirmatively identify amounts of a produced gas from a geologic formation that are of biogenic origin.

The following definitions are provided to clarify their specific use in the context of the invention.

“Biogenic feedstock” refers to organic material generated by living organisms or a product (e.g., a waste product) generated by living organisms or derived from organic material generated by living organisms. The term “biogenic feedstock” is intended to distinguish from fossil fuels and feedstocks derived from fossil fuels.

“Biogenic methane” or “biomethane” refers to methane (CH₄) that is generated from raw materials of biogenic origin (e.g., a biogenic feedstock). Biomethane is contrasted with “fossil methane” or “ancient methane,” which refers to fossil fuel methane or methane in which the carbon therein is of geologic age, typically derived from a hydrocarbon-bearing geologic formation or generated from other hydrocarbons extracted from a hydrocarbon-bearing geologic formation. Biomethane and renewable methane are further distinguished from methane where the carbon is derived from fossil carbon (e.g., fossil fuels), but is generated using energy from renewable resources (e.g., solar energy).

“Geologic formation” refers to a body of rock below the surface of the Earth which may be distinct from other neighboring bodies (e.g., based on type of rock). Geologic formations also include regions within a body of rock that contain or house carbonaceous materials, such as coal, oil, or natural gas, or that previously contained or housed such carbonaceous materials prior to extraction. Geologic formations include those having a porosity and permeability sufficient to store and transmit fluids, such as liquids or gases, which may be referred to herein as “subterranean reservoirs.” In some examples, hydrocarbons (e.g., fossil fuels) can be extracted from some geologic formations and such geologic formations can become depleted or include depleted regions, which can provide storage regions for injecting other material into the geologic formation, such as to store the other material (e.g., for long-term or short-term storage).

“Consortium of symbiotic microorganisms” refers to a mixture of various microorganisms living in a particular environment (e.g., in a geologic formation). In some examples, a consortium of symbiotic microorganisms comprises methanogens, which produce methane under anaerobic conditions from a carbonaceous feedstock.

“Radioisotope analysis” refers to refers to a process where a relative abundance of a radioisotope is evaluated for one or more chemical species. Radioisotope analysis is useful for tracking specific atoms in a chemical reaction or for tracking amounts of chemical species of different origin in a mixture.

“Stable isotope analysis” refers to a process where relative abundances of different stable isotopes of an element are evaluated. Stable isotope analysis can be performed for a single chemical species (e.g., CH₄) to determine amounts of different stable isotopes present in the single chemical species (e.g., amounts of ¹³C and ¹²C, in the case of CH₄). In some examples, a stable isotope analysis can be performed for reactants and products of a chemical reaction to identify one or more isotopic fractionation factors.

“Isotopic fractionation factor” refers to a measure of the extent to which different isotopes of an element are favored by a chemical reaction or process.

As described above, isotopic analysis can be used to affirmatively identify amounts of a produced gas from a geologic formation that are of biogenic origin. Two different types of isotopic analysis are described herein, which can be used individually or together to evaluate the origin of a produced fuel. In a first example, radioisotope analysis can be used. In a second example, stable isotope analysis can be used. In some cases, both radioisotope analysis and stable isotope analysis are used together, such as for purposes of calibration or validation.

In some examples, radioisotope analysis is useful for distinguishing between carbon that is biogenic in origin from carbon that is of ancient origin (e.g., fossil carbon). Radioisotope analysis involves determining the relative abundance of a radioactive isotope of an element (e.g., ¹⁴C) in a sample. For example, the radioisotope ¹⁴C, which has a half-life about 5700 years, is generated in Earth's atmosphere by interaction of nitrogen atoms with cosmic rays, so there is generally always some amount of ¹⁴C in Earth's biosphere, living organisms, and biomass, due to the uptake of CO₂ from the atmosphere by plants. In contrast, carbon that is fixed underground for a long duration (e.g., fossil carbon) tends to be depleted (e.g., completely depleted, even in the youngest fossil carbon deposits) in ¹⁴C because the age of the carbon is far in excess of the half-life of ¹⁴C or many multiples of the half-life of ¹⁴C. Thus, when a gas of biogenic origin (e.g., biomethane) is mixed with the same gas of ancient origin (e.g., fossil methane), radioisotope analysis can be used to determine the relative amounts of the gas in the mixture that are contributed from each.

For example, any of the gas that contains ¹⁴C can be attributed to a biogenic origin. Knowing the abundance of ¹⁴C that is present in the biogenic feedstock used to generate the gas (e.g., by methanogenic digestion), the same relative abundance of ¹⁴C is expected to be in the gas that is of biogenic origin. As an example, CO₂ in the atmosphere may have an abundance of ¹⁴C of about 1 part per trillion, and modern biogenic feedstocks will have essentially an identical abundance. Biofuels (e.g., biomethane) derived from such a biogenic feedstock will similarly have an identical abundance of ¹⁴C, when pure.

In the case of methane produced from a geologic formation where a consortium of symbiotic microorganisms is present that digests a biogenic feedstock injected into the formation, if the abundance of ¹⁴C in the produced methane is about 1 part per trillion, then about 100% of the produced methane can be determined to be of biogenic origin, for example. In another example, if the abundance of ¹⁴C in the produced methane is about 0.1 parts per trillion, then about 10% of the produced methane can be determined to be of biogenic origin, while about 90% of the produced methane can be determined to be of fossil origin. It will be appreciated that fractionation factors representing the relative rate at which ¹⁴C undergoes chemical reactions as compared to the stable isotopes ¹³C and ¹²C can be used to provide a more precise measure of the amount of a produced gas that is of biogenic origin.

Although radioisotope analysis of ¹⁴C can be used to determine an amount or fraction of a produced gas that is of biogenic origin, radioisotope analysis is complex, time-consuming, and expensive. Other analytic techniques, such as stable isotope analysis, may be less-complex, less time-consuming, and less expensive, but these techniques may not be as accurate as radioisotope analysis of ¹⁴C.

Stable isotope analysis involves determining the relative abundance of stable isotopes of an element (e.g., ¹²C and ¹³C) in a sample. Although the relative abundances of ¹²C and ¹³C in nature are about 98.89% ¹²C and 1.11% ¹³C, the relative abundances of ¹²C and ¹³C in a sample can be different. For example, when reactions involving ¹²C-containing species and similar ¹³C-containing species occur, the rates of reactions for the ¹²C-containing species and similar ¹³C-containing species can be different. In some cases, although the reaction rates may vary only slightly, a measurable change in the relative ¹²C and ¹³C abundances between reactants and products can occur. The differences in relative ¹²C and ¹³C abundances between a starting material (e.g., a biogenic feedstock) and a final product (e.g., biomethane) can be impacted by various factors, such as the number of reaction steps, the anaerobic digestion pathway(s) used, the isotopic content of the biogenic feedstock, etc. Further, the relative ¹²C and ¹³C abundances of fluids in the geologic formation (e.g., fossil methane), which may mix with the final products (e.g., biomethane) may contribute to the relative ¹²C and ¹³C abundances of produced fluid from the formation. Although carbon isotopes are described, relative abundances of other stable isotopes (e.g., ¹⁶O and ¹⁸O) can be similarly analyzed.

Upon determining the relative abundances of stable isotopes in the biogenic feedstock, and the produced gas, and optionally of any fossil origin gas in the geologic formation (e.g., determined prior to injecting the biogenic feedstock), this information can be combined with that obtained from the radioisotope analysis as a verification of the abundance analysis of the produced gas. For example, the stable isotope abundance in the produced gas may be impacted by contributions from gas of biogenic origin and of gas of fossil origin, and the relative amounts of the gas of biogenic origin and of gas of fossil origin obtained by a radioisotope analysis can provide calibration factors for the stable isotope analysis. In some examples, these calibration factors can be used in a subsequent analysis where the stable isotope analysis is used to determine the relative amounts of the gas of biogenic origin and of gas of fossil origin without having to a repeat radioisotope analysis.

It will be appreciated that the techniques described herein do not rely on isotopic tracers or isotopic labeling, such as where an artificially enhanced isotopic distribution of particular chemical species are introduced into a system, as described in U.S. Patent Application Publication No. 2012/0036923, hereby incorporated by reference. For example, isotopic tracers have been used in some cases to track the conversion of fossil carbon to fossil methane in a geologic formation. Instead, the techniques described herein make use of natural isotopic distributions in biogenic carbon to differentiate from fossil carbon, which has a distinct isotopic distribution. The techniques described here purposefully make use of biogenic feedstocks, which have different naturally occurring isotopic abundances of carbon than fossil carbon, for generation of biomethane. This biomethane exhibits an isotopic signature characteristic of the biogenic feedstock, which is different from an isotopic characteristic of fossil-derived methane.

It will further be appreciated that the techniques described herein are not just a matter of isotopically differentiating biogenically generated gaseous products from background gas present in coal reservoirs and tracing migration of biogas in coal reservoirs for purposes of mapping, as described in U.S. Patent Application Publication No. 2012/0036923. Instead, the techniques described herein make use of biogenic feedstocks for generation of biogenic gases within a geologic formation and subsequent production of the gases and are useful for identifying amounts of the produced gases that are biogenic in origin based on radioisotope and/or stable isotope characters of the biogenic feedstock and the produced gas without requiring specific isotope analysis of gases or fluids present in the geologic formation before introduction of the feedstock and generation of the biogenic gases. For example, specific embodiments of the disclosed techniques do not use or require isotopic analysis of background gas present in the geologic formation. The disclosed techniques can employ both stable isotope analysis and radioisotope analysis, with the radioisotope analysis optionally being used for purposes of calibrating or standardizing the stable isotope analysis, such as to estimate or determine measures of isotopic fractionation. The combination of stable isotope analysis and radioisotope analysis described provides a simplified, efficient, and highly reliable way to determine whether and how much of a gas produced from a geologic formation is of biogenic origin, providing advantages over stable isotope analysis or radioisotope analysis alone.

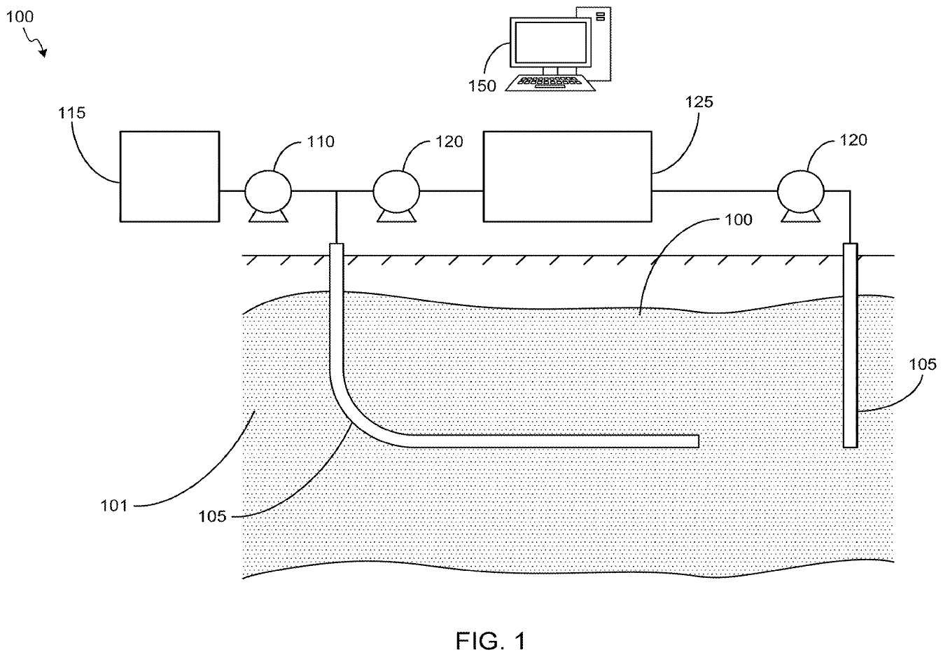

FIG. 1 provides a schematic illustration of an example system 100 showing a geologic formation 101 containing a consortium of symbiotic microorganisms. One or more wells 105 are shown in the geologic formation 101. The one or more wells 105 are shown in fluid communication with one or more pumps 110 for injecting a biogenic feedstock from a storage container 115 into the geologic formation 101. The one or more wells 105 are also shown in fluid communication with one or more pumps 120 for producing a fluid from the geologic formation 101. The produced fluid may be, at least temporarily directed to a storage tank 125 for further processing, storage, distribution, analysis, or the like.

In some examples, sampling ports (not shown) may be included in the storage container 115 or storage tank 125, or elsewhere in the system, to allow samples of the biogenic feedstock and/or the produced fluid to be obtained so that this material can be subjected to a radioisotope analysis and/or a stable isotope analysis. In some examples, radioisotope analysis and/or stable isotope analysis may be performed on manually obtained samples of fluid produced from the geologic formation 101 to determine isotopic distributions these samples, but automated sampling can also be used, such as where samples of the produced fluid are obtained periodically using a robotic, automated, or remotely controlled sampling system. Similarly, in some examples, isotope analysis may be performed on manually obtained samples of biogenic feedstock to determine isotopic distributions for the biogenic feedstock, but automated sampling can also be used, such as where samples of the biogenic feedstock are obtained periodically using a robotic, automated, or remotely controlled sampling system. Isotope analysis may be performed on site where the sample of the produced fluid is obtained, or it may be performed remotely, such as at a remote lab or other analysis facility. The isotope analysis can use any suitable analytical techniques or systems including, but not limited to, cavity ring-down spectroscopy, accelerator mass spectrometry, etc. In some examples, stable isotope analysis and radioisotope analysis can be performed on a single sample, but different samples can also be used for stable isotope analysis and radioisotope analysis.

System 100 further is shown including a computing device 150, which may include one or more processors and a non-transitory computer readable storage medium comprising instructions that, when executed by the one or more processors, cause the one or more processors to perform operations. In some examples, the instructions and/or operations may comprise operations that configure the computing device to identify an isotopic distribution for stable isotopes and/or radio isotopes included in the biogenic feedstock, identify an isotopic distribution for the stable isotopes and/or radio isotopes included in the gaseous product in the fluid, and determine an amount of the gaseous product in the fluid derived from the biogenic feedstock. In some examples, operations corresponding to identifying the isotopic distribution for stable isotopes and/or radio isotopes in the gaseous product in the fluid and/or in the biogenic feedstock can be performed by the system receiving the isotopic distribution(s), such as via one or more inputs or via one or more data or network communications. In some examples, the computing device 150 may be in data and/or control communication with one or more isotope analysis systems to control or receive isotopic distribution information from such systems.

The computing device 150 may optionally be in data or control communication with the one or more pumps 110 and/or the one or more pumps 120, such as to control and/or monitor injection of the biogenic feedstock or production of the fluid. As an example, results from one or more isotopic analyses may be used to determine operational decisions relating to injection of material into or production of material from the geologic formation 101. Operational decisions may, for example, include changing an injection rate, changing a production rate, altering a composition of the biogenic feedstock, or the like. For example, the biogenic feedstock may be provided as a component of an aqueous fluid injected into the geologic formation, and blend rates of the biogenic feedstock or other additives in the aqueous fluid (e.g., growth nutrients, trace elements, etc.) can be controlled. The computing device 150 may optionally be in data or control communication with one or more other sensors or control devices (not shown in FIG. 1), which can be used to monitor and/or control production rates, injection rates, blend amounts, or the like, such as according to one or more inputs or automated determinations, such as based on an isotopic analysis.

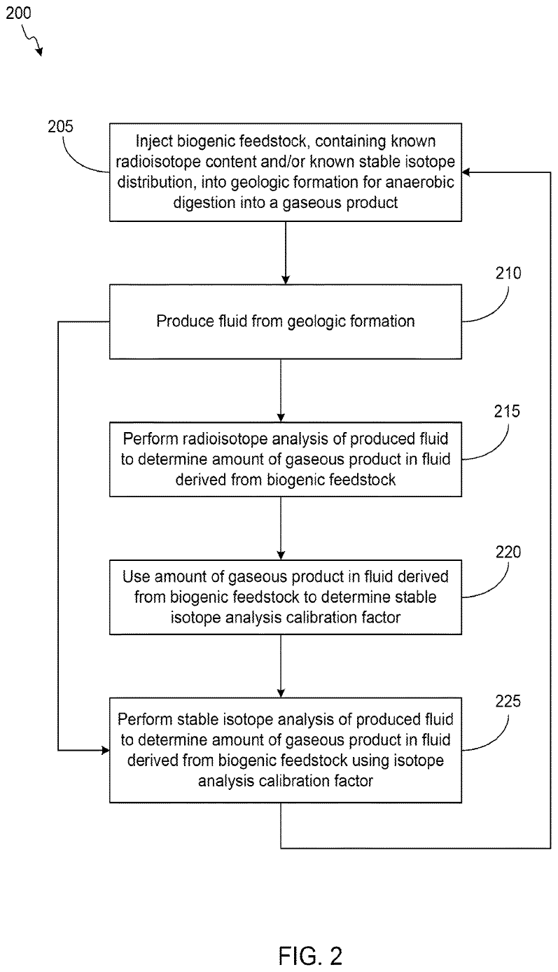

FIG. 2 provides an overview of example method 200 of producing and analyzing a fluid from a geologic formation according to various techniques described herein. Method 200 starts at block 205, where a biogenic feedstock is injected into a geologic formation. The geologic formation may include a consortium of symbiotic microorganisms that may anaerobically digest the biogenic feedstock, such as to generate a gaseous product (e.g., CH₄). In some examples, the geologic formation may comprise a coal bed formation or an oil field formation. The biogenic feedstock may contain a known radioisotope content (e.g., ¹⁴C) and/or a known distribution of stable isotopes (e.g., ¹²C and ¹³C). Depending on conditions in the geologic formation, anaerobic digestion may take some time, so method 200 may include allowing a suitable amount of time to pass (e.g., from 1 hour to 10 years).

At block 210, a fluid is produced from the geologic formation. The produced fluid may include the gaseous product (e.g., CH₄), of which at least a portion corresponds to that gaseous product (e.g., bio-CH₄) generated by digesting the biogenic feedstock by the consortium of symbiotic microorganisms. In some examples, the produced fluid may include a portion of the gaseous product that was not produced by the consortium of symbiotic microorganisms and/or may be of fossil origin (e.g., fossil CH₄). The produced fluid may optionally include other components, such as non-reactive or inert gases (e.g., N₂ or Ar), or other gaseous components present in the geologic formation (e.g., CO₂) or produced by the consortium of symbiotic microorganisms (e.g., H₂)

At block 215, a radioisotope analysis of at least a portion of the produced fluid (e.g., a gaseous portion) is performed, such as to determine a relative amount of the gaseous product in the produced fluid that is derived from the biogenic feedstock. The radioisotope analysis may include identifying an abundance of the same radioisotope that was included in the biogenic feedstock that is present in the gaseous product in the produced fluid. This abundance may be used to determine the relative amount of the gaseous product in the produced fluid that is derived from the biogenic feedstock.

At block 220, the relative amount of the gaseous product in the produced fluid that is derived from the biogenic feedstock is used to determine one or more stable isotope analysis calibration factors. The stable isotope analysis calibration factors can be generated in tandem with a stable isotope analysis of the produced fluid and the biogenic feedstock to determine how the stable isotopes in the biogenic feedstock partition in the relative amount of the gaseous product in the produced fluid that is derived from the biogenic feedstock.

It will be appreciated that blocks 215 and 220 of method 200 may not need to be performed for every implementation of method 200. In some examples, method 200 may branch from block 210 to block 225, where a stable isotope analysis of the produced fluid is performed to determine the relative amount of the gaseous product in the produced fluid that is derived from the biogenic feedstock, such as using the stable isotope analysis calibration factors determined at block 220.

In this way, the complex, time-consuming, and expensive process of radioisotope analysis can be performed a limited number of times, such as to determine one or more calibration factors, and/or as a periodic verification/recalibration, while the less complex, less time-consuming, and less expensive process of stable isotope analysis can be used, such as on a more regular or routine basis, to verify and/or track the relative amount of the gaseous product in the produced fluid that is derived from the biogenic feedstock.

In some cases, the relative amount of the gaseous product in the produced fluid that is derived from the biogenic feedstock can be used in a feedback mechanism to control the rate at which the biogenic feedstock, or other additives or components that are mixed into the biogenic feedstock, is injected into the geologic formation and/or the rate at which fluid is produced from the geologic formation.

The invention may be further understood by the following non-limiting examples.

### Example 1

Carbon is analyzed in produced samples by accelerator mass spectrometers (AMS), which determine the ratio of the three different carbon isotopes (¹⁴C/¹³C/¹²C) contained within the sample. At the end of an AMS run, data gathered is not only the number of carbon 14 atoms in the sample but also the quantity of carbon 12 and carbon 13. From these data, concentration ratio of the isotopes can be known to allow evaluation of the level of fractionation, which essentially is the pre-determined bias in the data due to mass effects. Once fractionation is corrected for, the sample's ¹⁴C is calibrated against an NBS (National Bureau of Standards) oxalic acid standard. The internationally accepted radiocarbon dating reference is 95% of the activity, in 1950 AD, of the NBS oxalic acid normalized to d¹³C of −13% with respect to Pee Dee Belemnite (PDB), the common normalization standard for stable gas isotope analysis. The factor of 0.95 adjusts the oxalic acid to the activity of wood from 1840 to 1860 (‘pre-industrial’). The radiocarbon fraction, or age, of the oxalic acid standard is determined as follows:

$${A}_{ON} = 0.95 {A}_{OX} [ 1 - 2 ( d {}^{13} C + 19 ) / 1000 ] .$$

A_{ON}: ¹⁴C activity of oxalic acid normalized for ¹⁴C fractionation.
A_{OX}: ¹⁴C activity of oxalic acid.
The d¹³C correction of 19% takes into account the fractionation of ¹⁴C during the combustion of oxalic acid.

Radiocarbon dating of produced samples: For produced water or gas studies, the pmc (percent modern carbon) notation is used:

$$pmc = ( {A}_{SN} / {A}_{abs} ) 100 \% = {{A}_{SN} [ {A}_{ON} {e}^{1 ( y - 1950 )} ]}^{- 1} 100 \% .$$

A_{SN}: activity of sample normalized for fractionation using d¹³C.
A_{ON}: ¹³C normalized activity of oxalic acid.
A_{abs}: Absolute ¹⁴C activity of the sample.
y: year of measurement of oxalic acid.

The resulting calculation of percent modern carbon quantifies the percentage of carbon-containing molecules that are modern, meaning derived from the carbon substrate that was injected.

A complicating factor in the analysis is the potential use of fossil carbon dioxide by methanogens via a hydrogenotrophic pathway. Table 1 depicts how carbon dioxide is both generated and consumed via the methanogenic fermentation of the model waste biomass compound glycerol:

TABLE 1

| | |
|---|---|
| Fermentation of glycerol to acetate | C₃H₈O₃ in water→ C₂H₄O₂+ CO₂ + 3H₂ |
| and hydrogen | |
| Aceticlastic methanogensis | C₂H₄O₂ → CH₄ + CO₂ |
| Hydrogenotrophic methanogensis | 4H₂ + CO₂ →CH₄ + H₂O |
| Net reaction | C₃H₈O₃ in water → 1.75 CH₄+ CO₂ + H₂O |

While the methanogenic fermentation of 1 mole of glycerol yields 1.75 moles of methane, any CO₂ consumed during hydrogenotrophic methanogenesis is likely to be derived from the large pool of aqueous CO₂ generated over geologic time, thus containing no carbon 14. This means that under this feedstock condition the apparent age of methane created solely from renewable carbon will be 57% modern (1/1.75).

### Example 2

Both stable isotope analysis and radioisotope analysis have advantages and disadvantages. Stable isotope analysis is rapid and inexpensive, yet requires repeated calibration using lab systems to accurately evaluate the volumes of new methane generation. Radioisotope analysis can provide a direct measure of renewable elements in a feedstock, yet is encumbered by slow and expensive analyses. To exploit the inherent values of each analysis, a calibration between the two can be performed, where both radioisotope and stable isotope analyses are performed on the same samples, leading to a constant or relational formula where radiocarbon results can be predicted from stable isotope results.

### Example 3

An alternate method to determine the conversion of renewable biomass to renewable carbon is the use of stable isotopes. Chemical bonds formed with heavier stable isotopes are stronger than those formed with lighter isotopes (e.g., ¹³C vs. ¹²C, ²H vs ¹H, etc.); therefore, the microbial conversion of feedstocks containing lighter elements to methane will preferentially occur. Since fossil carbon is fixed in place, and has been for geologic time, any historical biological conversion of these feedstocks will be biased towards the consumption of lighter isotopes, leaving heavier stable isotopes in place. As an example, when newly introduced renewable carbon is introduced into the system, it still contains the natural Carbon-13 isotope composition (1.1%), vs. fossil carbon, which will contain a greater percentage of Carbon-13 due to the historical microbial consumption of Carbon-12.

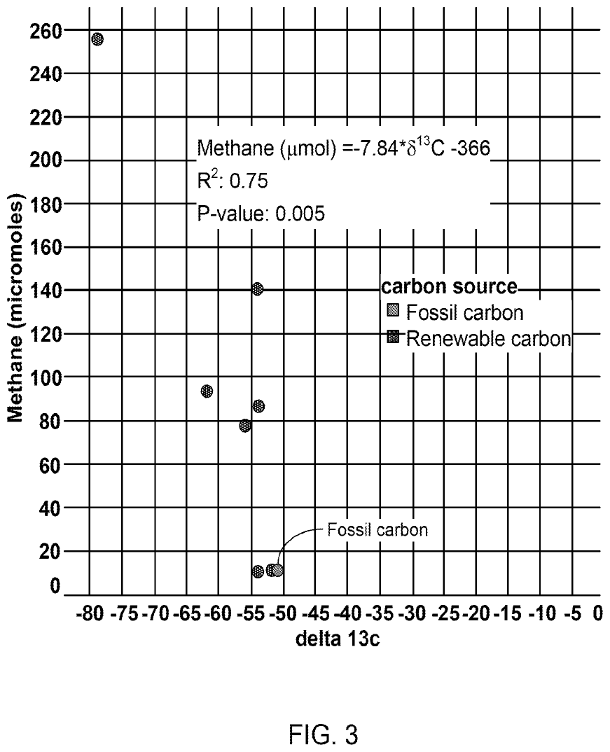

Laboratory data (obtained using cavity ring-down spectroscopy), depicted in FIG. 3, indicate that as methane is generated in field derived produced fluid samples fed different feedstocks, there is a strong linear relationship between the volume of methane created and the stable carbon isotope composition of the resulting methane gas. The control sample (red) identifies the delta ¹³C signature of fossil carbon, while samples noted in blue correspond to methane generated using renewable feedstocks and produced fluid consortia. This linear relationship can be used to quantify the mass of new methane created based on the gas isotope composition. As this relationship is generally dependent on environmental characteristics, it can be empirically determined in a laboratory setting for each reservoir targeted for feedstock injection.

### Example 4

This Example describes isotopic analysis measurements for determination of a stable isotope analysis calibration factor and subsequent use for determining the amount of a gaseous product generated by anaerobic digestion that is biogenic in origin. When expressed as a fractional or relative amount, the amount of a gaseous product generated by anaerobic digestion that is biogenic in origin is also referred to as percent modern carbon (pmc).

Samples of methane gas generated by anaerobic digestion are obtained. In some cases, the samples are mixed with some amount of fossil methane as a diluent. The samples are subjected to stable isotope analysis to determine delta (δ¹³C) value for the samples. For example, a ratio of ¹³C to ¹²C within the samples is determined and compared to the ratio of ¹³C to ¹²C in a standard. As a standard, comparison with a ratio of ¹³C to ¹²C for Pee Dee Belemnite (PDB) limestone is used. The delta value for the sample is obtained using the formula:

$$δ = [ \frac{{[ \frac{{}^{13} C}{{}^{12} C} ]}_{sample}}{{[ \frac{{}^{13} C}{{}^{12} C} ]}_{standard}} - 1 ] \times 1000.$$

Delta values are typically expressed in % or parts per 1000, thus the above formula includes a multiplication by 1000.

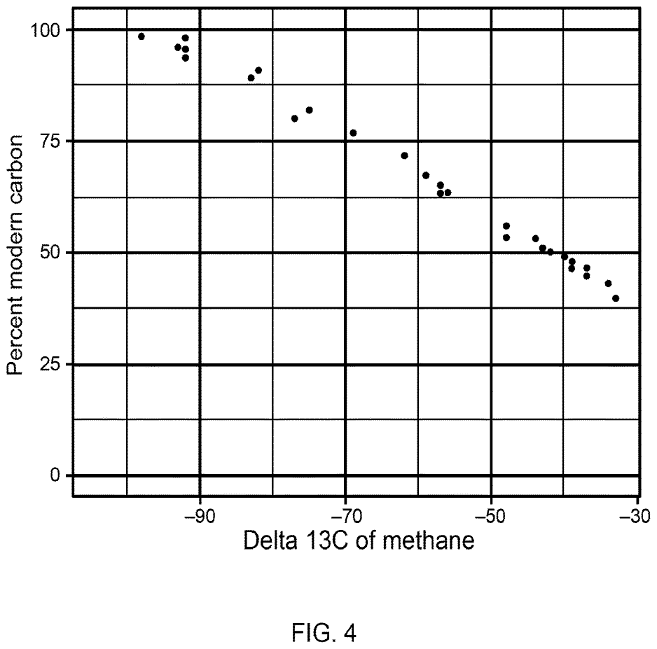

Radioisotope analysis for the samples is also performed, as described with respect to Example 1 above, to determine an absolute measure of the percent modern carbon (pmc) for each sample. With the pmc measurements and the δ¹³C measurements, the values can be referenced against one another to determine stable isotope analysis calibration factors. FIG. 4 provides a plot depicting example results of pmc determined by radioisotope analysis versus δ¹³C determined by stable isotope analysis.

In the present example, a clear relationship is observed and regression analysis or other analysis of the data can be performed to determine the stable isotope analysis calibration factors—in this case the regression constants output by the regression analysis, along with the type of regression used.

Once the stable isotopic analysis calibration factors are determined, the process described above for determining stable isotopic measurements of additional samples can be performed and used in combination with the stable isotopic analysis calibration factors to rapidly determine the amount of a sample of a gaseous product generated by anaerobic digestion that is biogenic in origin (pmc), optionally along with an uncertainty as warranted by the regression analysis. Table 2 provides example values for additional δ¹³C measurements and the corresponding pmc values output using the stable isotopic analysis calibration factors. A considerable advantage can be gained by using stable isotopic analysis results in this way to determine pmc values since stable isotope analysis measurements are considerably less complex and less time consuming as compared to radioisotope analysis, which can allow for real-time or near real-time determination of the amount of biogenic carbon in gas produced from a geologic formation.

TABLE 2

| δ¹³C (‰) | Percent Modern Carbon (%) |
|---|---|
| −37 | 46 |
| −55 | 62 |
| −76 | 81 |

### REFERENCES

U.S. Patent Application Publication No. 2012/0036923, Valentine “Tracer Method to Estimate Rates of Methane Generation Through Augmentation or Biostimulation of the Sub-Surface,” Feb. 16, 2012.
Palstra et al., 2014, “Biogenic Carbon Fraction of Biogas and Natural Gas Fuel Mixtures Determined with ¹⁴C,” Radiocarbon, Vol 56, No 1, pp. 7-28, DOI: 10.2458/56.16514
Taguchi et al., “Biobased carbon content of resin extracted from polyethylene composite by carbon-14 concentration measurements using accelerator mass spectrometry” SpringerPlus, 3:6, DOI: 10.1186/2193-1801-3-6

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2 and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given, are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by examples, embodiments, and optional features, modification and variation of the concepts herein disclosed may be resorted to by those skilled in the art, and that such modifications and variations are considered to be within the scope of this invention as defined by the appended claims.

What is claimed is:

1. A method, comprising:
injecting a biogenic feedstock into a geologic formation, the geologic formation containing a consortium of symbiotic microorganisms, wherein the consortium of symbiotic microorganisms at least partly anaerobically digests the biogenic feedstock to generate a gaseous product, wherein the biogenic feedstock contains a known amount of a radioisotope;
producing a fluid from the geologic formation, the fluid comprising the gaseous product; and
performing a radioisotope analysis of at least a portion the fluid, using the known amount of the radioisotope, to identify an amount of the gaseous product in the fluid derived from the biogenic feedstock.

2. The method of claim 1, further comprising:
using the amount of the gaseous product in the fluid derived from the biogenic feedstock to determine one or more stable isotope analysis calibration factors.

3. The method of claim 2, further comprising:
injecting a second biogenic feedstock into a second geologic formation, the second geologic formation containing a second consortium of symbiotic microorganisms, wherein the second consortium of symbiotic microorganisms at least partly anaerobically digests the second biogenic feedstock to generate the gaseous product;
producing a second fluid from the second geologic formation, the second fluid comprising the gaseous product; and
performing a stable isotope analysis of at least a portion of the second fluid, using the one or more stable isotope analysis calibration factors, to determine a second amount of the gaseous product in the second fluid derived from the second biogenic feedstock.

4. The method of claim 3, wherein the stable isotope analysis of at least a portion of the second fluid excludes, does not include, or is performed without measuring a baseline isotopic distribution for gases present in the second geologic formation prior to injecting the second biogenic feedstock.

5. The method of claim 3, wherein producing the second fluid is performed while or after injecting the second biogenic feedstock.

6. The method of claim 3, wherein the geologic formation and the second geologic formation are a same geologic formation.

7. The method of claim 2, wherein the one or more stable isotope analysis calibration factors include one or more isotopic fractionation factors or are characteristic of or derived from one or more isotopic fractionation factors.

8. The method of claim 2, wherein using the amount of the gaseous product in the fluid derived from the biogenic feedstock to determine the one or more stable isotope analysis calibration factors comprises:
determining an isotopic distribution for stable isotopes included in the biogenic feedstock;
determining an isotopic distribution for the stable isotopes included in the gaseous product in the fluid; and
determining the one or more stable isotope analysis calibration factors using:
the isotopic distribution for the stable isotopes included in the biogenic feedstock,
the isotopic distribution for the stable isotopes included in the gaseous product in the fluid, and
the amount of the gaseous product in the fluid derived from the biogenic feedstock.

9. The method of claim 8, wherein the stable isotopes included in the biogenic feedstock include ¹²C or ¹³C, and wherein the stable isotopes included in the gaseous product include ¹²C or ¹³C.

10. The method of claim 1, wherein the radioisotope comprises ¹⁴C.

11. The method of claim 1, wherein the gaseous product comprises CH₄, CO₂, H₂, or any combination of these.

12. The method of claim 1, wherein the biogenic feedstock comprises waste glycerol, erythritol, sorbitol, or maltitol, wastewater, a waste product, a carbonaceous material source, or any combination of these.

13. The method of claim 1, wherein the radioisotope analysis of at least a portion of the fluid excludes, does not include, or is performed without measuring a baseline isotopic distribution for gases present in the geologic formation prior to injecting the biogenic feedstock.

14. The method of claim 1, wherein producing the fluid is performed while or after injecting the biogenic feedstock.

15. The method of claim 1, wherein the geologic formation is an oil field formation or a coal bed formation.

16. A method, comprising:
identifying one or more stable isotope analysis calibration factors;
injecting a biogenic feedstock into a geologic formation, the geologic formation containing a consortium of symbiotic microorganisms, wherein the consortium of symbiotic microorganisms at least partly anaerobically digests the biogenic feedstock to generate a gaseous product;
producing a fluid from the geologic formation, the fluid comprising the gaseous product; and
performing a stable isotope analysis of at least a portion of the fluid, using the one or more stable isotope analysis calibration factors, to determine an amount of the gaseous product in the fluid derived from the biogenic feedstock.

17. The method of claim 16, wherein the one or more stable isotope analysis calibration factors include one or more isotopic fractionation factors or are characteristic of or derived from one or more isotopic fractionation factors.

18. The method of claim 16, wherein identifying the one or more stable isotope analysis calibration factors comprises:
injecting a second biogenic feedstock into a second geologic formation, the second geologic formation containing a second consortium of symbiotic microorganisms, wherein the second consortium of symbiotic microorganisms at least partly anaerobically digests the second biogenic feedstock to generate the gaseous product, wherein the second biogenic feedstock contains a known amount of a radioisotope;
producing a second fluid from the second geologic formation, the second fluid comprising the gaseous product; and
performing a radioisotope analysis of at least a portion of the second fluid, using the known amount of the radioisotope, to determine the one or more stable isotope analysis calibration factors.

19. The method of claim 18, wherein the radioisotope analysis of at least a portion of the second fluid excludes, does not include, or is performed without measuring a baseline isotopic distribution for gases present in the second geologic formation prior to injecting the second biogenic feedstock.

20. The method of claim 18, wherein producing the second fluid is performed while or after injecting the second biogenic feedstock.

21. The method of claim 18, wherein the radioisotope comprises ¹⁴C.

22. The method of claim 18, wherein the geologic formation and the second geologic formation are a same geologic formation.

23. The method of claim 16, wherein performing the stable isotope analysis of at least a portion of the fluid comprises:
determining an isotopic distribution for stable isotopes included in the biogenic feedstock;
determining an isotopic distribution for the stable isotopes included in the gaseous product in the fluid; and
determining the amount of the gaseous product in the fluid derived from the biogenic feedstock using:
the isotopic distribution for the stable isotopes included in the biogenic feedstock,
the isotopic distribution for the stable isotopes included in the gaseous product in the fluid, and
the one or more stable isotope analysis calibration factors.

24. The method of claim 23, wherein the stable isotopes included in the biogenic feedstock include ¹²C or ¹³C and wherein the stable isotopes included in the gaseous product include ¹²C, or ¹³C.

25. The method of claim 16, wherein the gaseous product comprises CH₄, CO₂, H₂, or any combination of these.

26. The method of claim 16, wherein the biogenic feedstock comprises waste glycerol, erythritol, sorbitol, or maltitol, wastewater, a waste product, a carbonaceous material source, or any combination of these.

27. The method of claim 16, wherein the stable isotope analysis of at least a portion of the fluid excludes, does not include, or is performed without measuring a baseline isotopic distribution for gases present in the geologic formation prior to injecting the biogenic feedstock.

28. The method of claim 16, wherein producing the fluid is performed while or after injecting the biogenic feedstock.

29. The method of claim 16, wherein the geologic formation is an oil field formation or a coal bed formation.

30. A system, comprising:
one or more pumps in fluid communication with a geologic formation containing a consortium of symbiotic microorganisms, the one or more pumps configured to:
inject a biogenic feedstock into the geologic formation, wherein the consortium of symbiotic microorganisms at least partly anaerobically digests the biogenic feedstock to generate gaseous product, and
produce a fluid from the geologic formation, the fluid comprising the gaseous product; and
a computing device configured to:
identify an isotopic distribution for stable isotopes included in the biogenic feedstock,
identify an isotopic distribution for the stable isotopes included in the gaseous product in the fluid, and
determine an amount of the gaseous product in the fluid derived from the biogenic feedstock using:
the isotopic distribution for the stable isotopes included in the biogenic feedstock,
the isotopic distribution for the stable isotopes included in the gaseous product in the fluid, and
one or more stable isotope analysis calibration factors.

31. The system of claim 30, wherein the one or more stable isotope analysis calibration factors include one or more isotopic fractionation factors or are characteristic of or derived from one or more isotopic fractionation factors, or wherein the one or more stable isotope analysis calibration factors are determined using a radioisotope analysis technique.

32. The system of claim 30, wherein the gaseous product comprises CH₄, CO₂, H₂, or any combination of these, or wherein the biogenic feedstock comprises waste glycerol, erythritol, sorbitol, or maltitol, wastewater, a waste product, a carbonaceous material source, or any combination of these.

33. The system of claim 30, wherein the geologic formation is an oil field formation or a coal bed formation.
